(19) United States
(12) Patent Application Publication
(10) Pub. No.: US 2007/0142804 A1
(43) Pub. Date: Jun. 21, 2007

(54) Hollow-core fibers

(76) Inventor: Bobby Lynn Bernard, Rogersville, TN (US)

Correspondence Address: Tammye L. Taylor;Eastman Chemical Company, P.O. Box 511, Kingsport, TN, 37662-5075, US

(21) Appl. No.: 11/303,370

(22) Filed: Dec. 16, 2005

(51) Int. Cl. A61F 13/15

(52) U.S. Cl. 604/375; 604/374

(57) ABSTRACT

A hollow-core fiber that is composed of a phthalyl substituted cellulose ester polymer having a degree of substitution of phthalyl moieties of greater than about 0.13. The hollow-core fiber is useful as an absorbent or fluid transfer member in an absorbent device such as catamenial devices, bandages, diapers, incontinence articles, training pants, bed pads, sweat absorbing pads, skin cleansing pads, hard surface cleansing pads, shoe pads, helmet liners, absorbent devices for surgical and dental purposes, air and water filtration devices, and industrial spill and leak absorbents.

The present invention relates to fibers and particularly to hollow-core fibers. More particularly, the hollow-core fibers of the present invention exhibit an anti-microbial effect. The present invention further relates to structures and articles made from the fibers where an improved liquid handling capability and/or an anti-microbial effect would be desirable. The present invention further relates to structures and articles made from the hollow-core fibers wherein a secondary compound is contained within the hollow-core of at least a portion of the fibers and optionally, wherein the secondary compound is releasable from the hollow-core.

Absorbent articles, such as diapers, sanitary napkins, tampons, paper towels, and the like universally include an absorbent for acquiring liquid, semi-solid, or viscous materials. The purpose of the absorbent is to transport liquids from one location to another, and more specifically, from the point of impact, to the non-affected areas of the absorbent and to retain the fluids within the structure. This permits greater absorbent capacity utilization, less waste and desirably prevents leakage from the absorbent structure. Thus, one key performance criteria for an absorbent article is the ability at which aqueous or viscous materials are transported and stored, generally via capillary effects of the absorbent structures. An important consideration used in constructing such structures is the porosity of the materials comprising the absorbent article. There is prior art that has been directed at optimizing the porosity or pore size of fibrous structures, foamed structures, structures made from particulate materials or combinations thereof. As a result, in the art the creation of gradient structures wherein the absorbent, typically known as the “absorbent core”, exhibits different degrees of hydrophilicity and/or capillary transport is known.

For example, it is known to those skilled in the art to: 1) include such materials as superabsorbent polymers in the absorbent core; 2) provide a density gradient in at least one direction of the absorbent core, i.e., in the X, Y and/or Z directions; or 3) provide combinations of materials and density gradients. As used herein, the “X” direction lies within the plane of the absorbent article and it is along the machine direction or longitudinally, the “Y” direction lies within the plane of the absorbent article and is along the cross-machine direction or transversely, and the “Z” direction is generally orthogonal to both the “X” and “Y” directions and typically is the thickness of the absorbent. However, each of these methods is not without its problems. In the case where a superabsorbent polymer is added, the polymer may selectively absorb the aqueous portion of the contacting fluid. Additionally, superabsorbents are known to swell and thereafter impede further transfer of impinging fluids to the non-affected absorbent, particularly when the absorbent core is rewet with an impinging fluid. When employing a density gradient in the absorbent core, a balance between a lower density absorbent and a higher density absorbent must be followed. A higher density absorbent core improves distribution but sacrifices the fluid intake rate and a low density absorbent core improves fluid intake rate but sacrifices the fluid distribution and capacity.

Accordingly, in the area of absorbent structures there is a need for an absorbent fiber that can be incorporated into an absorbent core that will increase fluid intake and distribution.

Moreover, when the absorbent core will be subjected to blood or bodily discharges, it would be advantageous to include within the absorbent core a fiber having an antimicrobial effect.

### SUMMARY OF THE INVENTION

One aspect of the present invention is a hollow-core filament or staple fiber comprising a phthalyl substituted cellulose having a degree of substitution (DS) of phthalyl ester moieties greater than about 0.13.

Another aspect of the present invention is an absorbent structure or article made from the filament or staple fibers of the present invention.

Yet another aspect of the present invention is a structure or articles made from the hollow-core filament or staple fibers wherein a secondary compound is contained within at least a portion of the hollow-core in the fibers or at least a portion of the fibers. Optionally, the secondary compound may be released from the hollow-core upon a desired predetermined set of conditions, such as temperature or moisture.

It is an object of the present invention to provide a filament or staple fiber having a hollow-core configuration wherein the filament or staple fiber comprises a substituted cellulose ester having a DS of greater than about 0.13 of phthalate ester moieties or substituents.

It is another object of the present invention to provide an absorbent structure utilizing the hollow-core filament or staple fiber which exhibits an anti-microbial effect.

### BRIEF DESCRIPTION OF THE DRAWINGS

FIG. 1 is a scanning electron photomicrograph of a fiber of the present invention.

FIG. 2 is a scanning electron photomicrograph longitudinal cross-section of a fiber of the present invention.

FIG. 3 is an absorbent article, such as a catamenial or feminine pad, that includes fibers of the present invention.

FIG. 4 is an absorbent article, shown as a tampon, that includes fibers of the present invention.

### DETAILED DESCRIPTION OF THE INVENTION

In a first embodiment (A), the hollow-core filament or fiber of the present invention comprises a phthalyl substituted cellulose having a DS greater than about 0.13 of a phthalate ester substituent or residue on the cellulose moiety. As used herein, the term “degree of substitution”, “DS” or “DS/AGU” refers to the average number of acyl substituents per anyhydroglucose ring of the cellulose polymer where the theoretical maximum DS is 3. In the present invention, the substituted cellulose has a DS of from about 0.13 to about 1.3, for example , the DS is from about 0.26 to about 1.3, or from about 0.52 to about 1.04, or from about 0.78 to about 1.04. For the cellulose esters of this invention, the DS or DS/AGU may be determined using any method known in the art. For example, using proton NMR. DS can be determined by 1H NMR in d-6 dimethylsulfoxide (DMSO) or tetrahydrofuran (THF) containing several drops of trifluoroacetic acid (to shift any hydroxyl protons downfield), or in tetrachloroethane containing several drops of trifluoroacetyl isocynate, or by hydrolysis of a sample of the cellulose ester followed by quantification of liberated carboxylic acids by gas chromatography. As used herein, the term “staple fiber” refers to a fiber, either natural or synthetic, that has been cut from a filament and for the sake of brevity, the terms will hereinafter be used interchangeably, unless specified or understood to be otherwise. One skilled in the art will understand that the balance of the acyl substituents per anhydroglucose ring of the cellulose polymer material comprising the hollow-core fiber can be any compatible secondary ester material and preferably is an ester having from two to twenty-four carbon atoms, and more preferably is selected from acetate, butyrate, isobutyrate, propionate, and mixtures thereof. A suitable material comprising the hollow-core fiber of the present invention has a phthalyl DS of about 0.92 and an acetyl DS of about 2.17 and is available from Eastman Chemical Company, Kingsport, Tenn.

In a second embodiment (B), the hollow-core fiber of the present invention can be prepared from a polymer blend having greater than about 75 weight % of a first polymer comprising the phthalyl substituted cellulose ester polymer described above. For example, the blend has greater than about 80 weight % of the first polymer, or greater than about 85 weight % of the first polymer, or the blend has even greater than about 95 weight % of the first polymer. As noted above, the phthalyl substituted cellulose polymer has a DS of from about 0.13 to about 1.3, for example from about 0.26 to about 1.3, or from about 0.52 to about 1.04, or about 0.78 to about 1.04, wherein the balance of substituted acyl moieties being an ester having from two to twenty-four carbon atoms, and are selected from one or more of acetate, butyrate, isobutyrate, or propionate.

The remainder of the polymer blend comprises a compatible second polymer or copolymer, wherein the total weight percentages of the first and second polymers in the blend equals 100. The second polymer is a polymer that is compatible with the first polymer and preferably is capable of being dissolved in a co-solvent and wet spun into a hollow-core fiber. Suitable secondary polymers include, but are not limited to, substituted cellulose esters wherein the substituted ester moiety has from two to twenty-four carbon atoms, and is selected from one or more of cellulose acetate, cellulose butyrate, cellulose isobutyrate, cellulose acetate butyrate, cellulose acetate isobutyrate, cellulose propionate, or cellulose acetate propionate.

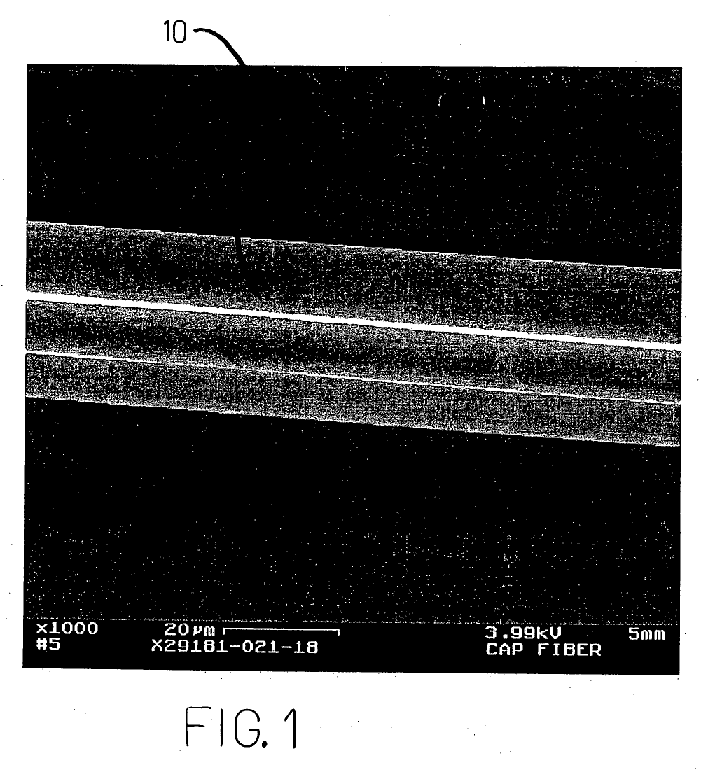

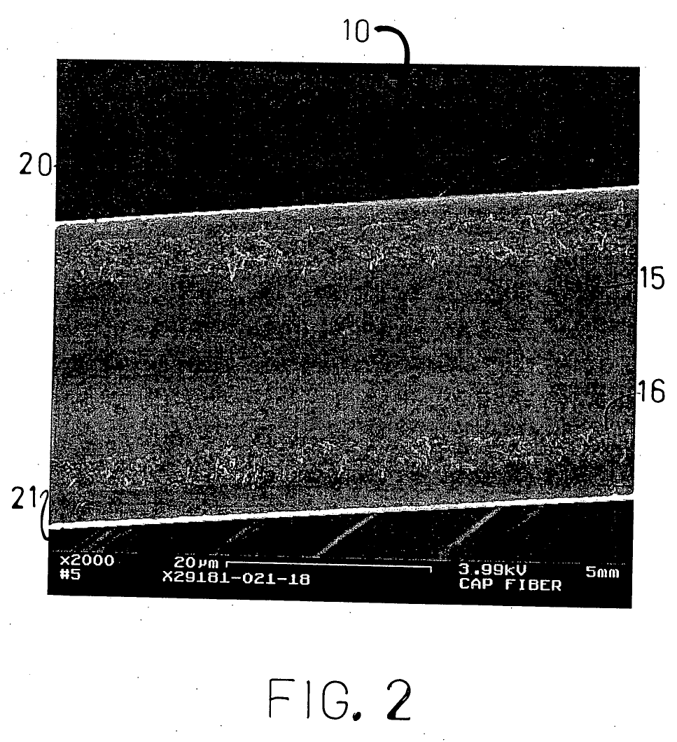

Referring to FIGS. 1 and 2, the hollow core staple fibers 10 of the present invention have a total diameter of from about 5 to about 1000 microns, for example from about 10 to about 500 microns, or from about 15 to about 100 microns and or from about 15 to about 85 microns. The hollow-core fiber length can be from about 0.1 millimeters to about 10 centimeters or it can be from about 1 millimeter to about 15 millimeters. Referring to FIG. 2, the hollow-core fiber 10 has an inner wall 15 that circumferentially defines an inner open or void area of the hollow-core fiber 10. The inner opening diameter is determined by the distance from one side 15 of the inner wall to an opposing side 16 of the inner wall, and generally is from about 5 to about 85 percent of the total diameter of hollow-core fiber. As used herein, the term “total diameter” refers to the diameter across the fiber determined by distance from a first point 20 on an outer wall to a directly opposing second point 21 on the other outer wall. The inner opening diameter is from about 25 to about 75 percent of the total diameter of hollow-core fiber 10 or the inner opening diameter is from about 50 to about 75 percent of the total diameter of hollow-core fiber. The percentage of hollowness of the hollow-core fiber can be determined in different ways. In one exemplary technique, the fiber is transversely cut (substantially perpendicular to the fiber) to form a cross-section of the fiber. The diameters of the opening and total diameter are then measured. Another technique is to cut the fiber substantially longitudinally or along the length of the fiber and then measure the inner and outer diameters of the fiber.

Additionally, a plasticizer can be used to improve the water resistance of the cellulose acetate phthalate fiber. Suitable plasticizers include one or more of acetylated monoglyceride, butyl phthalylbutyl glycolate, dibutyl tartrate; diethyl phthalate, dimethyl phthalate, ethyl phthalyethyl glycolate, glycerin, propylene glycol, triacetin, triacetin citrate, and tripropionin. Other additives conventionally used in the production of polymeric filaments and fibers can also be incorporated into the polymer. Such additives include, but are not limited to, UV stabilizers, pigments, delusterants, lubricants, antistatic agents, and water and alcohol repellents. The additives may be added in conventional amounts, which is typically less than about 10 weight % based on weight of the fiber.

The polymeric filament of the present invention is prepared using the wet solution spinning process or solvent spinning process, which is well known to those skilled in the art. Generally, the cellulose acetate phthalate is dissolved in a suitable solvent, such as acetone, typically about 20 weight % polymer and 80 weight % solvent. In a solvent spinning process the polymeric solution is pumped through the spinneret which may be submerged in a liquid bath in which the solvent is soluble to solidify the polymeric fibers. The spinneret is suspended so that the polymeric filament exiting the spinneret is contacted with a counter-current or co-current hot air stream to evaporate the solvent. After spinning, the filaments are attenuated by withdrawing the filament from the spinning device at a speed that is faster than the extrusion speed. The attenuated filaments are taken up on rotating nip rolls for subsequent processing.

In another embodiment of the present invention, the hollow-core fiber may contain an auxiliary compound selected from absorbents, odor controlling or absorbing materials, fragrances and perfumes, medicaments, therapeutic agents and mixtures thereof in which one or more may be adapted to be releasably incorporated into the inner portion of the hollow-core fiber. Hollow-core fibers having auxiliary compounds included in the inner portion can be used in the fabrication of a variety of absorbent articles described in greater detail below.

Auxiliary fluid absorbents, such as a superabsorbent polymer, are known to those skilled in the art. As used herein “superabsorbent” refers to a water-swellable, water insoluble organic or inorganic material capable of absorbing at least about 15 times its weight in an aqueous solution containing 0.9 weight % sodium chloride. Organic materials suitable for use as a superabsorbent material in conjunction with the present invention include, but are not limited to, natural materials such as guar gum, agar, pectin and the like; as well as synthetic materials, such as synthetic hydrogel polymers. Such hydrogel polymers include, for example, alkali metal salts of polyacrylic acids, polyacrylamides, polyvinyl alcohol, ethylene, maleic anhydride copolymers, polyvinyl ethers, methyl cellulose, carboxymethyl cellulose, hydroxypropylcellulose, polyvinylmorpholinone, and polymers and copolymers of vinyl sulfonic acid, polyacrylates, polyacrylamides, polyvinylpyrridine, and the like. Other suitable polymers include hydrolyzed acrylonitrile grafted starch, acrylic acid grafted starch, and isobutylene maleic anhydride polymers and mixtures thereof. The hydrogel polymers are preferably lightly crosslinked to render the materials substantially water insoluble. Crosslinking may, for example, be accomplished by irradiation or by covalent, ionic, van der Waals, or hydrogen bonding. Superabsorbents are generally available in particle sizes ranging from about 20 to about 1000 microns. Examples of suitable commercially available superabsorbents are SANWET IM 3900 available from Hoescht Celanese located in Portsmouth, Va. and DRYTECH 2035LD available from Dow Chemical Co. located in Midland, Mich. The superabsorbent polymer, if used, is incorporated into at least a portion of the void space of the hollow-core fiber using techniques known to those skilled in the art, such as by immersing at least one end of the fiber in a solution containing the polymer which would thereafter be dried.

The hollow-core fiber may include an auxiliary odor control substance, a releasable fragrance, or a combination of both in the opening or void area of the fiber. Suitable odor controlling material can be any material known to those skilled in the art that will effectively control, neutralize and/or adsorb odor. Preferably, the odor controlling material is a solid material. Examples of such materials include, but are not limited to, baking soda (sodium bicarbonate), activated charcoal, activated carbon, clays, diatomaceous earth, zeolites and mixtures thereof. Typically, on a percentage basis, the odor controlling material is generally present in an amount of about 0.5 to about 400% by weight of the hollow-core fiber. If the amount of the odor controlling material is below about 0.5% by weight, effective odor control may not be achieved. Therefore, the odor controlling material is between about 1% and about 40% by weight of the weight of the hollow-core fiber, or between about 5% and about 20% by weight of the weight of the hollow-core fiber. Depending of the basis weight of the absorbent layer incorporating the hollow-core fiber of the present invention, the loading of the odor controlling material is between about 2 gsm and about 80 gsm. For example, the loading of the odor controlling material is between about 8 gsm and about 40 gsm or between about 12 gsm and 30 gsm.

The odor controlling material may be incorporated into the hollow-core fiber by methods known to those skilled in the art. For example, the odor controlling material may be prepared in situ in the hollow-core fiber by impregnating the fiber with liquid soda ash which is then converted to sodium bicarbonate by immersing the treated fibers in humid carbon dioxide. Other similar methods could be used to incorporate the odor controlling material in the hollow-core fiber.

The hollow-core fiber may also contain an optional releaseable fragrance or perfume to provide a positive scent signal to a consumer during use of the absorbent article. Perfume may be incorporated in the hollow-core fibers at a level of from about 0.005% to about 0.20 weight %, or from about 0.01% to about 0.15 weight %, or from about 0.01% to about 0.08 weight %, or even from about 0.03% to about 0.06 weight % of the hollow-core fiber. Examples of such fragrances or perfumes include volatile, hydrophilic materials; volatile, hydrophobic materials; and low odor detection threshold perfume materials.

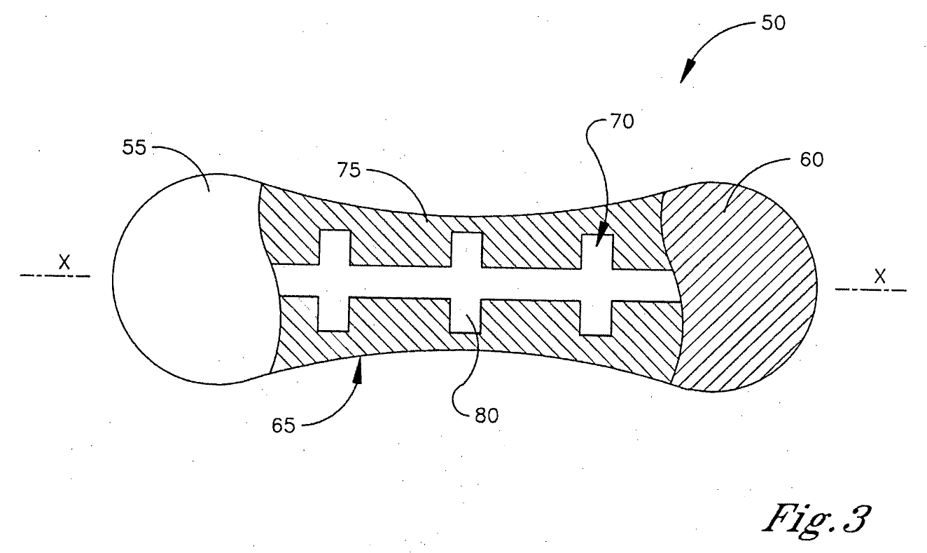

Referring to FIG. 3, an absorbent article utilizing the absorbent fibers of the present invention is illustrated as a catamenial absorbent article 50. Such devices are often used by women to absorb the flow of body fluids, such as menses, blood, urine, and other excrements. Typically, the absorbent article includes a liquid-permeable cover 55 disposed adjacent to the wearer, a liquid-impermeable baffle 60 generally positioned adjacent to the undergarment, and an absorbent core 65 sandwiched between the cover 55 and the baffle 60. A wide variety of staple fibers materials can be employed in the absorbent structures of the present invention. For example, staple fibers may be formed from cellulosic fibers such as wood pulp, and modified cellulose fibers, textile fibers and substantially nonabsorbent synthetic polymeric fibers.

The liquid-permeable cover 55 is sanitary, clean in appearance, and somewhat opaque to hide bodily discharges collected in and absorbed by the absorbent core 65. The cover 55 further exhibits good strike-through and rewet characteristics permitting bodily discharges to rapidly penetrate through the cover 55 to the absorbent core 65, but not allow the body fluid to flow back through the cover to the skin of the wearer. For example, some suitable materials that can be used for the cover 55 include nonwoven materials, perforated thermoplastic films, or combinations thereof. A nonwoven fabric made from polyester, polyethylene, polypropylene, bicomponent, nylon, rayon, or like fibers may be utilized. For instance, a white uniform spunbond material is particularly desirable because the color exhibits good masking properties to hide menses that has passed through it.

If desired, the cover 55 may also be sprayed with a surfactant to enhance liquid penetration to the absorbent core 65. The surfactant is typically non-ionic and should be non-irritating to the skin. Additionally, the cover 55 can also contain a plurality of apertures (not shown) to permit body fluid to pass more readily into the absorbent core 65. The apertures can be randomly or uniformly arranged throughout the cover 55, or they can be located only in the narrow longitudinal band or strip arranged along the longitudinal axis X-X of the absorbent article 50. The apertures permit rapid penetration of body fluid down into the absorbent core 65. The size, shape, diameter and number of apertures can be varied to suit one's particular needs.

The absorbent core 65 includes a fluid transfer member 70 that is in liquid communication with a fluid storage member 75. As used herein, “fluid communication” means that fluid can transfer readily between two absorbent components or layers (e.g., the fluid transfer layer and the storage layer) without substantial accumulation, or restriction by an interposed layer. For example, tissues, nonwoven webs, construction adhesives, and the like can be present between the two distinct components while maintaining “fluid communication”, as long as they do not substantially impede or restrict fluid as it passes from one component or layer to another. The fluid transfer member 70 is shown as a longitudinal strip having a plurality of transversely extending members 80 for fluid transfer and distribution to the fluid storage member 75. One skilled in the art would understand that the fluid transfer member 70 can be any configuration that achieves the purpose of fluid transfer and distribution. The fluid transfer member 70 comprises from about 5 to about 100 weight % of at least one of the hollow-core staple fibers (A) and/or (B) of the present invention. For example, the fluid transfer member 70 comprises from about 50 to about 100 weight % or from about 95 to about 100 weight % of the hollow-core staple fibers (A) and/or (B) of the present invention. The fluid transfer member 70 resides adjacent to the cover 55 with at least a portion of the fluid transfer member 70 centrally positioned over the fluid storage member 75.

The fluid storage member 75 can be any absorbent material known to those skilled in the art. For example, one type of absorbent material is a coformed air-laid composite of melt spun synthetic filaments intermingled with staple natural fibers. Any of a variety of synthetic polymers may be utilized as the melt-spun component of the coform material. For example, suitable thermoplastics include polyolefins, such as polyethylene, polypropylene, polybutylene; polyamides; or polyesters, with polypropylene being preferred. Suitable staple fibers include polyester, rayon, cotton, with pulp fibers being preferred. Pulp fibers are generally obtained from natural sources such as woody and non-woody plants. Woody plants include, for example, deciduous and coniferous trees. Non-woody plants include, for example, cotton, flax, esparto grass, milkweed, straw, jute, and bagasse. Wood pulp fibers typically have lengths of about 0.5 to about 10 micrometers and a length-to-maximum width ratio of about 10/1 to about 400/1. A typical cross-section has a width of about 30 micrometers and a thickness of about 5 micrometers. Coforming processes are described in greater detail in U.S. Pat. Nos. 4,118,531 and 4,100,324 the entire disclosures of which are incorporated herein by reference. Generally, the manufacturing of coform absorbent material uses a forming apparatus having a meltblowing unit and a movable, forming wire belt. The meltblowing apparatus has a die head through which air streams pass. A supply device delivers the polymer to an extruder for delivering melted polymer to the die head. The melted polymer leaves the extruder die head in fine polymer streams and is combined with a primary air stream. The fine polymer streams leaving the die head are attenuated by the converging flows of high velocity heated gas supplied through nozzles which break the polymer streams into discontinuous microfibers of small diameter. The resulting microfibers have an average fiber diameter of up to about 10 microns, often averaging about 5 microns. While the microfibers are predominantly discontinuous, they generally have a length exceeding that normally associated with pulp fibers. The primary air stream is merged with a secondary air stream containing individualized pulp fibers so as to integrate the two different fibrous materials in a single step. The individualized wood pulp fibers typically have a length of about 0.5 to about 10 micrometers. Optionally, the hollow-core fibers of the present invention may also be added to the individualized pulp fibers for integrating the hollow-core fibers into the fluid storage member 75. The secondary air stream having the pulp fibers is then placed onto the forming belt that passes beneath the forming die as the polymer microfibers and the air streams are directed downwardly. The forming belt may be provided with suction boxes that withdraw air from beneath the forming belt and provide for a uniform layer of fibers onto the belt.

Other methods of incorporating the hollow-core fiber of the present invention with other absorbent fibers into a fibrous matrix are well known to those skilled in the art. Suitable methods include concurrent air-laying and/or wet-laying the hollow-core fibers of the present invention with the other absorbent fibers. Alternatively, the hollow-core fibers of the present invention can be intermixed with an absorbent matrix batt which is subsequently compressed to the desired density or thickness. Other methods include sandwiching the hollow-core fibers between two layers of absorbent materials so that the hollow-core fibers allow fluid communication between the two layers.

It is further recognized that the absorbent structure incorporating the hollow-core fibers can be used in the absorbent structure as a separate layer, zone or area within a larger, composite structure, or the hollow-core fibers can be combined with other absorbent materials, layers, or structures. Methods for affixing two or more separate layers together are well known in the art and include adhesives, melt bonding or wrapping two or more separate layers in as separate absorbent material such as tissue or crepe paper.

As stated above, the absorbent article also includes a baffle 60. The baffle 60 is generally liquid-impermeable and designed to face the inner surface, i.e., the crotch portion of an undergarment (not shown). The baffle 60 can permit a passage of air or vapor out of the absorbent article 50 while still blocking the passage of liquids. Any liquid-impermeable material can generally be utilized to form the baffle 60. For example, one suitable material that can be utilized is a microembossed polymeric film, such as polyethylene or polypropylene. In particular embodiments, a polyethylene film is utilized that has a thickness in the range of about 0.2 mils to about 5.0 mils, and particularly between about 0.5 to about 3.0 mils. Generally, in forming the absorbent article 50, the cover can surround the absorbent core 65 so that it completely encases the absorbent core or, alternatively, the cover 55 and baffle 60 extend beyond the absorbent core 65 and are then bonded together at the periphery. The cover 55 and baffle 60 can be joined using ultrasonic bonding, adhesives or any other method known to those skilled in the art.

The absorbent article 10 may also contain other components as well. For instance, in some embodiments, the lower surface of the baffle 60 can contain an adhesive for securing the absorbent article 10 to an undergarment. In such instances, a releaseable backing, such as silicone coated Kraft paper, may be utilized to protect the adhesive so that the adhesive remains clean prior to attaching the absorbent article to undergarment.

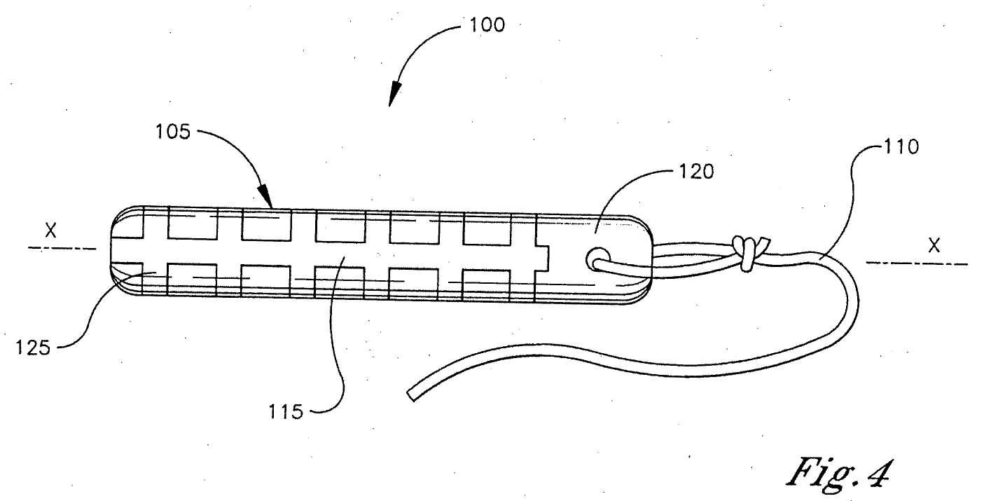

Referring to FIG. 4, another embodiment of the present invention is illustrated as a tampon 100. The tampon is an absorbent article primarily designed to be worn by a woman during her menstrual period to absorb menses, blood, and other body fluids. A tampon may be also worn by a woman during other phases of the menstrual cycle if a medicament is incorporated into the hollow-core fibers, as would be the case in the invention described herein if the symptoms or conditions to be treated manifest themselves at a time other than during her menstrual period.

The tampon 100 includes an absorbent structure or core 105 with a withdrawal string 110. The absorbent structure 105 is normally compressed into the form of a cylinder and can have a blunt, rounded or shaped forward end that is closer to the cervix when the tampon 100 is in use. The absorbent structure 105 also has a proximal end that is closer to the vaginal opening when the tampon 100 is in use. The tampon 100 commonly has a withdrawal string 110 fastened to the proximal end that serves as a means for withdrawing the tampon from the woman's vagina. The withdrawal string 110 can be looped through an aperture formed transversely through the absorbent structure 105. In addition, the withdrawal string 110 can have a knot formed at the free end of the string to assure that the string will not separate from the absorbent structure 105.

The absorbent core 105 includes a fluid transfer member 115 in liquid communication with a fluid storage member 120. The fluid transfer member 115 is shown as a longitudinal strip having a plurality of transversely extending members 125 for fluid transfer and distribution to the fluid storage member 120. One skilled in the art would understand that the fluid transfer member 115 can be any configuration that achieves this purpose of fluid transfer and distribution. For example, the fluid transfer member 115 may comprise a separate layer (not shown) encasing the fluid storage member 120. The hollow-core fibers of the present invention comprises from about 5 to about 100 weight % of the fluid transfer member 115. For example, the hollow-core fibers comprise from about 50 to about 100 weight % or from about 95 to about 100 weight % of the fluid transfer member 115. It is also preferred that at least a portion of the fluid transfer member 115 be centrally positioned over the fluid storage member 120. The basis weight of the fluid transfer member 115 can be from about 0.1 grams per square meter (gsm) to about 5000 gsm, but preferably is from about 5 gsm to about 100 gsm.

The fluid storage member 120 is an absorbent typically formed from fibers that are assembled into an absorbent sheet or ribbon. Alternatively, the fluid storage member 120 can be formed from absorbent fibers that are assembled and compressed into a generally elongated and/or cylindrical configuration. The fluid storage member 120 is desirably formed from cellulosic fibers, such as cotton and rayon. For example, the fluid storage member 120 can be 100% cotton, 100% rayon, a blend of cotton and rayon fibers, or other materials known to be suitable for tampons, including artificial fibers such as polyester, polypropylene, nylon or blends thereof. The fluid storage member 120 may also include degradable fibers. Other types of materials or structures may also be used, such as cellulose sponge or a sponge formed from elastomeric materials. When formed, the fluid storage member 120 typically includes interstitial space or voids between the fibers or other materials.

It is further known to those skilled in the art for the tampon to include a permeable cover sheet. Suitable cover stock includes a sheet formed from spunbond fibers of a hydrophobic polymeric material, e.g., a spunbond polypropylene.

One issue associated with tampon use is menstrually occurring toxic shock syndrome which is a sometimes fatal, multi-system disease associated with the infection or colonization by Staphylococcus aureus (S. aureus) bacteria. It is believed by some researchers that tampons provide an increased surface area for the S. aureus bacteria to grow and adequate oxygen for toxin production. Accordingly, to reduce the occurrence of menstrually occurring toxic shock syndrome manufactures of tampons have coated at least a portion of the tampon with one or more bacteriological inhibitory compounds. For example, U.S. Pat. No. 5,389,374 issued to Brown-Skrobot in Feb. 14, 1995, discloses using glyceryl monolaurate to lower the TSST-1 toxin production from S. aureus bacteria.

However, it has been discovered that fibers of the present invention advantageously exhibit anti-microbiological effect.

In the examples below, the specified test material was assessed for antimicrobial effectiveness after inoculation with a test organism and then evaluated to determine the percent reduction of the test organism after specified exposure periods. The antimicrobial effectiveness of the present invention was evaluated using test method 100-2004 as specified by the Technical Manual of the American Association of Textile Chemists and Colorists, (AATCC) vol. 78, 2003 as modified herein.

The sample material for Examples 1-14 is as specified below. Methods and procedures for preparing cellulose acetate phthalate are well known to those skilled in the art. For all the examples, except for Examples 7 and 14, the amount of water in the fiber dope was 1 weight % at the time of spinning. For Examples 7 and 14 the amount of water in the fiber dope was 3 weight % at the time of spinning. However, from the data presented below, this did not appear to affect the biocidal efficiency.

The cellulose acetate phthalate fibers were produced from powder cellulose acetate phthalate NF having a phthalyl DS of 0.92 (available from Eastman Chemical Company, Kingsport, Tenn.). The dry powder was dissolved in acetone or acetone/water and spun into fibers having a denier of 4.3, 5.1 and 6.1 on an apparatus as described in U.S. Pat. No. 3,077,633. As used herein, the term “denier” is a unit of weight measurement equal to one gram per 9000 meters of filament length.

Filaments were cut into fibers having a length of ⅛ to ¼ inches by hand using a manual cutting board, and separated into individual fibers by mixing in a Warner Blender using a non-cutting blade.

To prepare the fibers into a mat structure, 2.4 grams of the specified fiber are weighed into a container and diluted with demineralized water. The mat structures were then produced as described in the TAPPI method T 205 om-88 (1988), beginning with procedure 7.2 “sheetmaking”, and the entire disclosure of which is incorporated herein by reference. The mat structures were then dried as described in Section 7.6 at 200° C. The resulting mat structures were approximately 6 inches (15.2 cm) in diameter and about 0.45 to 0.50 mm thickness. The mat structures were also washed free of any remaining residual acetone from the spinning process.

The specified micronized powder was produced by cryogenic grinding the material to an average diameter of less than 22 micron. The mat structures containing micronized powder were prepared by adding the powder to the water wet mat structures prior to drying. From 0.45 of a gram to about 0.50 of a gram of micronized powder was added to each mat structure containing the micronized powder. The micronized powder was thermally fused to the fibers by hot pressing the mat structure at a temperature of 180° C. to 200° C. for 30 minutes using an Emerson Speed Dryer, Model 135, available from Emerson Apparatus of Portland, Me.

Examples 1, 3, 8 and 10 were composed of cellulose acetate phthalate (CAP) fibers having a denier of 4.3 and approximately one-eighth (0.32 cm) to one-quarter (0.64 cm) of an inch in length. The fibers are available from Eastman Chemical Company, Kingsport, Tenn., U.S.A.

Examples 2, 5, 9 and 12 were composed of cellulose acetate phthalate fibers having a denier of 5.1 and approximately one-eighth (0.32 cm) to one-quarter (0.64 cm) of an inch in length. Intermixed with the cellulose acetate phthalate fibers was one-half of a gram of cellulose acetate phthalate powder having an average diameter of less than 22 microns. The cellulose acetate phthalate powder and fibers and are available from Eastman Chemical Company, Kingsport, Tenn., U.S.A.

Examples 4 and 11 were composed of cellulose acetate phthalate fibers having a denier of 5.1 and approximately one-eighth (0.32 cm) to one-quarter (0.64 cm) of an inch in length. The fibers are available from Eastman Chemical Company, Kingsport, Tenn., U.S.A.

Examples 6 and 13 were composed of cellulose acetate phthalate fibers having a denier of 5.1 and approximately one-eighth (0.32 cm) to one-quarter (0.64 cm) of an inch in length. Intermixed with the cellulose acetate phthalate fibers was one-half of a gram of cellulose acetate powder having an average diameter of less than 22 microns. The cellulose acetate powder and fibers and are available from Eastman Chemical Company, Kingsport, Tenn., U.S.A.

Examples 7 and 14 were composed of cellulose acetate phthalate fibers having a denier of 5.1 and approximately one-eighth (0.32 cm) to one-quarter (0.64 cm) of an inch in length. The fibers are available from Eastman Chemical Company, Kingsport, Tenn., U.S.A.

### EXAMPLES 1-7

The materials specified in Table 1 below were tested using 3 inch (7.6 cm) swatches of material exposed to an aerosol challenge of Staphylococcus aureus (ATCC #6538) which was repeatedly delivered to each test material over a two minute interval. The technique was modified from NLI standard BFE test to provide a challenge level of greater than 1×10⁶ colony forming units (CFU)/test article. The flow rate through the test article was maintained at 30 L/min. (1.1 cubic feet /min (CFM)). The face velocity, determined by the flow rate divided by the surface area, was maintained at 22 feet per minute (6.7 meters/min) unless specified otherwise.

In preparing the aerosol challenge of Staphylococcus aureus, 100 ml of soybean casein was inoculated with the bacterium and incubated at 37° C. (±2° C.) for 24 hours (±4 hours) with mild shaking. An amount of the inoculated soybean casein was diluted with peptone water to achieve an aerosol challenge concentration of greater than 1×10⁶ CFU. The challenge procedure was run as follows. Tubing was connected to a nebulizer and run through a peristaltic pump and into the challenge containing vessel. The lines to the nebulizer were then purged. The peristaltic pump was calibrated to deliver a constant challenge volume throughout the testing interval of 2 minutes. The test system was then allowed to equilibrate by running 2-3 blanks or unused control samples. Aliquots (30 mL) of peptone water were placed into an all glass impinger (AGI) for a challenge titer run. The challenge titer was conducted under standard test conditions to determine the concentration of challenge aerosol droplets being delivered to the test articles. The flow rate of the challenge aerosol was maintained at 30 L/min. Allowing the nebulizer to run through a peristaltic pump, aliquots of 30 ml of the inoculant were delivered to a test vessel for 1 minute and then turned off. The vacuum and pressure were allowed to run for 1 additional minute to clear the nebulizer and glass aerosol chamber of excess aerosol particles and afterwards it was turned off. Standard plate count procedures were used to determine the titer of the control and a six-stage Andersen sampler was used to determine the mean particle size of the aerosol. To inoculate each test sample, the test sample was placed in the sample holder and the challenge procedure repeated except that no AGI was used. The titer of the AGI assay fluid was determined using standard plaque assay procedures.

Immediately following the 2 minute challenge, the test article was removed from the apparatus and placed in a closed containment vessel maintained at a temperature of 37° C. (±2° C.) for the designated time intervals. At each sampling interval, the inoculated swatch was placed in a flask containing approximately 100 mL of Letheen broth or other neutralizer(s) as needed. The flask was then manually shaken for approximately 1 minute. The neutralizer was serially diluted as necessary and evenly spread on a soybean casein digest agar (SCDA) plate using a sterile bent glass rod. The plates were then incubated at 37° C. (±2° C.) for 48-72 hours or until the colonies could be counted.

A neutralization control was performed using uninoculated treated samples of each type in 100 mL aliquots. Approximately 100-10,000 CFU/mL of the Staphylococcus aureus was added to the extract fluid. The aliquots were then placed onto SCDA. Titer of the diluted Staphylococcus aureus was confirmed by adding the same volume of inoculum to a 100 mL bottle of Letheen broth or other neutralizer(s). The plate aliquots were then incubated at 37° C. (±2° C.) for 48-72 hours or until the colonies could be counted.

The organism counts are specified below as CFU/specimen sample, i.e., test swatch. The percent reduction was then determined.

Positive and negative controls for the test organism were also maintained. The positive control consisted of a 100 mL bottle of neutralizer spiked with the challenge organism. The negative control consisted of a sterile 100 mL bottle of neutralizer.

The results of the test are presented in Table I below. For Samples 1 and 2, the average control titer at time 0 was 1.4×10⁶ CFU and Samples 3-7 the average control titer at time 0 was 3.3×10⁶ CFU. Counts shown as approximate ( [symbol] )) were taken from results where data was found outside the range of 25-250 CFU. Counts shown as (<) or (>) are due to calculations which included at least one instance of less than 1 CFU recovery. Negative (−) percent reductions demonstrate an ending titer that was greater than the starting titer.

TABLE I

| Sample | Exposure | Recovered | Percent |
|---|---|---|---|
| ID | Interval (hrs.) | (CPU) | Reduction |
| 1 | 0 | 1.3 × 10⁶ | 11 |
| | 24 | 1.8 × 10⁴ | 98.7 |
| | 48 | [symbol] 4.6 × 10⁴ | [symbol] 96.8 |
| 2 | 0 | 1.2 × 10⁶ | 15 |
| | 24 | [symbol] 1.7 × 10⁴ | [symbol] 98.8 |
| | 48 | <3.3 × 10² | >99.98 |
| 3 | 0 | 9.6 × 10⁵ | 71 |
| | 24 | [symbol] 1.3 × 10⁴ | 99.62 |
| | 48 | <2.3 × 10³ | >99.3 |
| 4 | 0 | 1.4 × 10⁶ | 56 |
| | 24 | 9.4 × 10³ | 99.72 |
| | 48 | [symbol] 5.1 × 10³ | [symbol] 99.84 |
| 5 | 0 | 1.1 × 10⁶ | 66 |
| | 24 | <2.0 × 10² | >99.99 |
| | 48 | [symbol] 2.2 × 10³ | [symbol] 99.93 |
| 6 | 0 | [symbol] 6.5 × 10⁵ | [symbol] 80 |
| | 24 | [symbol] 4.7 × 10³ | [symbol] 99.86 |
| | 48 | <2.0 × 10² | >99.99 |
| 7 | 0 | 1.2 × 10⁶ | 63 |
| | 24 | [symbol] 6.4 × 10³ | [symbol] 99.8 |
| | 48 | [symbol] 9.0 × 10² | [symbol] 99.97 |

### EXAMPLES 8-14

In examples 8-14 the general procedures as set forth above for Examples 1-7 were following, with the following exceptions. The challenge organism was bacteriophage phi-X174 (ATCC #13706B1) incorporated into Escherichia coli (E. coli C, ATCC #13706), a coliform as the host for the virus.

To prepare the phi-X174 bacteriophage, approximately 100 mL of a nutrient broth was inoculated with E. coli C and incubated for about 6-18 hours at 37° C. (±2° C.) with stirring at about 200-250 rpm. A 1:100 dilution of the E. coli C culture was prepared and incubated at 37° C. (±2° C.) with stirring at about 200-250 rpm to grow a culture having a density of 2-4×10⁸ CFU/mL. This density corresponded to an optical density of 0.3-0.5 on a spectrophotometer at 640 nanometers.

The E. coli C bacteria culture was then inoculated with 5-10 mL of the bacteriophage phi-X174 so that the ratio of bacteriophage to bacteria cells would be between 0.1 to 2.0. The mixture was incubated for about 1 to 5 hours at 37° C. (±2° C.) with stirring at about 100-250 rpm. The mixture was then centrifuged at 10,000×G for about 20 to 40 minutes. The supernatant was then filtered through a sterile 0.2 m filter to remove the host cell debris and the phage stock was recovered. The test culture was then grown in a nutrient broth at 37° C. (±2° C.) for 18 to 24 hours.

Following the challenge procedure for Examples 1-7 above, various samples were subjected to a 2 minute challenge. Immediately following the 2 minute challenge, the test article was removed from the apparatus and placed in a closed containment vessel maintained at a temperature of 20-25° C. for the designated time intervals. At each sampling interval, the inoculated swatch was placed in a flask containing approximately 100 mL of Letheen broth or other neutralizer(s) as needed. The flask was then manually shaken for approximately 1 minute.

The plaque assay procedure was performed as follows. Two and one-half milliliters of molten top agar was dispensed into sterile test tubes and held at 45° C. (±2° C.) in a water bath. Aliquots of 0.5 mL of the appropriate dilutions were added to the top agar test tubes. One to two drops of the E. coli C bacterial culture was added to each test tube. The contents were mixed well and poured over the surface of bottom agar plates. The agar was allowed to solidify and then incubated at 37° C. (±2° C.) for 6-18 hours; the length of time was selected to provide plaques that were large enough but not merging.

A neutralization control was performed using uninoculated treated samples of each type in 100 mL aliquots. Approximately 100-10,000 PFU/mL of the E. coli C bacteria culture was added to the extract fluid. Titer of the diluted E. coli C bacteria culture was confirmed by adding the same volume of inoculum to a 100 mL bottle of Letheen broth or other neutralizer(s).

The organism counts are specified below as PFU/specimen sample, i.e., test swatch. The percent reduction was then determined. The results of the test are presented in Table II below. The average control titer at time 0 was 8.4×10⁵ PFU for Samples 12-15, and 1.1×10⁶ for Samples 16-22, unless specified otherwise. Counts shown as approximate ( [symbol] ) were taken from results where data was found outside the range of 25-250 PFU. Negative (−) percent reductions demonstrate an ending titer that was greater than the starting titer.

TABLE II

| Sample | Exposure | Recovered | Percent |
|---|---|---|---|
| ID | Interval (hrs.) | (PFU) | Reduction |
| 8 | 0 | 4.3 × 10⁵ | 48 |
| | 24 | 1.4 × 10⁵ | 83 |
| | 48 | [symbol] 4.0 × 10⁴ | [symbol] 95.2 |
| 9 | 0 | 2.7 × 10⁵ | 68 |
| | 24 | 9.6 × 10⁴ | 88 |
| | 48 | 9.8 × 10⁵ | −17 |
| 10 | 0 | 7.2 × 10⁵ | 35 |
| | 24 | 9.0 × 10⁵ | 18 |
| | 48 | [symbol] 4.0 × 10⁵ | [symbol] 64 |
| 11 | 0 | 3.0 × 10⁵ | 72 |
| | 24 | 7.5 × 10⁵ | 31 |
| | 48 | 3.1 × 10⁵ | 72 |
| 12 | 0 | 7.2 × 10⁵ | 34 |
| | 24 | 3.4 × 10⁵ | 69 |
| | 48 | 6.1 × 10⁵ | 45 |
| 13 | 0 | 1.0 × 10⁶ | 7 |
| | 24 | [symbol] 4.5 × 10⁵ | [symbol] 59 |
| | 48 | 2.9 × 10⁵ | 74 |
| 14 | 0 | 6.5 × 10⁵ | 41 |
| | 24 | 6.6 × 10⁵ | 40 |
| | 48 | 7.5 × 10⁵ | 32 |

### EXAMPLES 15-17

In the following Examples, tests were conducted in multiples of 5 samples each and the average of the absorbency reported. In performing the test, fiber samples weighing approximately 5±0.05 grams were placed in a 316 stainless steel basket having dimensions of 23 mm in diameter, 37 mm deep and weighting 9.003 grams. The basket was held above the surface of the water, at a temperature of 25 °±1° C., at a distance of about 12 mm. The basket was lowered into the water and allowed to completely submerge. The basket was then removed from the water and allowed to drain for 10 seconds. The basket and fibers were then weighed. The weight of the test basket and fibers was deducted from this weight to determine the amount of water absorbed by the fibers. The results are in Table III below.

TABLE III

| Example No. | Fiber denier | % Weight Gain |
|---|---|---|
| 15 | 4.3 | 1980 |
| 16 | 5.1 | 1978 |
| 17 | 6.1 | 1888 |

As can be seen from Table III, the hollow-core fibers of the present invention have a significant absorptive capacity.

In accordance with another aspect of the present invention, the hollow-core fibers utilized in the absorbent structures described above can include auxiliary materials selected from medicament or therapeutic agents (herein after used interchangeably), incorporated on and preferably inside of the hollow-core fiber. This may include hydrophilic (aqueous based), hydrophobic (oil or lipid based), or slurries (liquid in combination with solid particles). Still referring to FIG. 4, the therapeutic agent may be applied to the hollow-core fibers using conventional methods for applying a therapeutic agent to the desired absorbent article. For example, unitary tampons may be dipped directly into a bath having the agent in a solution or slurry the liquid portion of which later evaporates upon drying, leaving the solid drug particles behind. The therapeutic agent or a formulation containing the therapeutic agent may be applied after the tampon is compressed.

The therapeutic agent, when incorporated on and/or into the hollow-core fibers, may be fugitive, loosely adhered, bound, or any combination thereof. As used herein the term “fugitive” means that the therapeutic agent is capable of migrating from the fiber. Alternatively, the therapeutic agent may be applied directly onto an individual layer comprising the hollow-core fibers before the layer is incorporated into the absorbent product.

Active ingredients, such as pharmaceutical compounds (e.g., histidines, anti-inflammatory drugs, calcium or potassium channel blockers), antimicrobials, anti-viral agents, antifungal agents, anti-metabolites, steroids, anesthetics, hormones or hormone inhibitors, pH control agents, and the like, can be included in the hollow-core fibers in any known drug delivery medium that is placed within the tampon. An example is micro-encapsulation of the active ingredient in starch, dextran, or other degradable or soluble materials, so that the microcapsules placed in the absorbent material of the tampon can permit gradual release of the active ingredient upon wetting, an increase in temperature, or physical contact. Another type of delivery system is the use of polymeric transport systems; these systems absorb materials and will release these materials when applied to a substrate.

Preferred therapeutic agents are those that will be absorbed into a user's body through the vaginal epithelium. Alternatively, or in addition, therapeutic and other beneficial agents such as vitamins, moisturizers, pro-biotic agents that promote the growth of normal vaginal bacterial flora, and the like may be similarly delivered. Therapeutic agents for use in the invention are absorbable through the vaginal epithelium and travel to the uterus by a unique portal of veins and arteries which are known to exist between the vagina, the cervix and the uterus. This anastomosis eliminates so called first pass metabolism by the liver, effectively delivering higher concentrations of therapeutic agent to the uterus than would otherwise be available via oral dosing. One skilled in the art knows the efficacy of therapeutic agents in such an application when introduced at a particular anatomical location. For example, when the therapeutic agent is selected to treat dysmenorrhea, it preferably is selected from the group consisting of nonsteroidal anti-inflammatory drugs (NSAIDs), prostaglandin inhibitors, COX-2 inhibitors, local anesthetics, calcium channel blockers, potassium channel blockers, β-adrenergic agonists, leukotriene blocking agents, smooth muscle inhibitors, and drugs capable of inhibiting dyskinetic muscle contraction.

COX-2 inhibitors, such as Celecoxib, Meloxicam, Rofecoxib, and Flosulide are novel anti-inflammatory and analgesic compounds. These compounds effectively inhibit production of COX-2 enzyme that is induced by pro-inflammatory stimuli in migratory cells and inflamed tissue. Because COX-2 is also involved in reproductive processes, selective COX-2 inhibitors will reduce uterine contractions in pre-term labor and relieve painful uterine contractions associated with dysmenorrhea by blocking prostaglandin receptors in the uterus. Additionally, they may reduce endometrial bleeding.

Suitable NSAIDs include Aspirin, Ibuprofen, Indomethacin, Phenylbutazone, Bromfenac, Sulindac, Nabumetone, Ketorolac, Mefenamic Acid, and Naproxen. Suitable local anesthetics include Lidocaine, Mepivacaine, Etidocaine, Bupivacaine, 2-Chloroprocaine hydrochloride, Procaine, and Tetracaine hydrochloride. Suitable calcium channel antagonists include Diltaizem, Israpidine, Nimodipine, Felodipine, Verapamil, Nifedipine, Nicardipine, and Bepridil. Examples of potassium channel blockers include Dofetilide, E-4031, Imokalant, Sematilide, Ambasilide, Azimilide, Ted isamil, RP58866, Sotalol, Piroxicam, and Ibutilide. Examples of suitable β-adrenergic agonists include Terbutaline, Salbutamol, Metaproterenol, and Ritodrine. Vasodilators, which are believed to relieve muscle spasm in the uterine muscle, include nitroglycerin, isosorbide dinitrate, and isosorbide mononitrate.

Examples of beneficial botanicals may include, but are not limited to, Agnus castus, aloe vera, comfrey, calendula, dong quai, black cohosh, chamomile, evening primrose, Hypericum perforatum, licorice root, black currant seed oil, St. John's wort, tea extracts, lemon balm, capsicum, rosemary, Areca catechu, mung bean, borage seed oil, witch hazel, fenugreek, lavender, and soy. Vaccinium extracts commonly derived from many members of the heath family, cranberries such as blueberries, and azaleas (Rhododendron spp.) as well as from red onion skin and short and long red bell peppers, Beta vulgaris (beet) root extract, and capsanthin may also be used. Other berries that have applicability are whortleberry, lingenberry, chokeberry, sweet rowan, rowanberry, seabuckhornberry, crowberry, strawberries, and gooseberries.

These beneficial therapeutic agents promote epithelial health in the vaginal region by delivering botanical ingredients with a feminine care device. The idea is to modulate the vaginal environment to enhance the wellness of this anatomical region. These benefits can be rather simple, for example increasing comfort by providing moisturization and/or lubricity. These benefits can also be more complex, for example modulating epithelial cell function to address vaginal atrophy. The beneficial therapeutic agents may reduce negative sensations such as stinging, burning, itching, etc, or introduce positive sensations to improve comfort.

For example, many therapeutic benefits have been ascribed to a large number of different botanical preparations. Preparations may include water-in-oil emulsions, oil-in-water emulsions, gel, liquid, dispersion, powder, and anhydrous systems, ointment, or salve, such as a botanical oil in an anhydrous base (e.g., petrolatum), or polyethylene glycol based systems. Also, botanicals are often prepared or extracted under conditions to generate water-soluble or oil-soluble extracts. These extracts are usually compositionally different and may have different skin and vaginal health benefits. Processing conditions will have an effect on the type of formulation that can be used and this will restrict the type of botanical (water or oil type) selected. Therefore, wide ranges of botanicals have utility in this invention. Botanicals can possess a variety of actives and activities that can include, but are not necessarily limited to, analgesics, antimicrobials, pro-biotic agents, anti-inflammatory compounds, anti-virals, enzymes, enzyme inhibitors, enzyme substrates, enzyme cofactors, ions, ion chelators, lipids, lipid analogs, lipid precursors, hormones, inflammatory mediators, inflammatory agonists, oxidants, antioxidants, humectants, growth factors, sugars, oligosaccharides, polysaccharides, vasodilators, and potential combinations thereof. It is understood that, for the purposes of this invention, the botanicals can be combined with any number of non-botanical active ingredients as well, including, but are not limited to, vitamins, calcium, magnesium, hormones, analgesics, prostaglandin inhibitors, prostaglandin synthetase inhibitors, leukotriene receptor antagonists, essential fatty acids, sterols, anti-inflammatory agents, vasodilators, chemotherapeutic agents, and agents to treat infertility.

It may not be necessary to impregnate the entirety of hollow-core fibers of an absorbent product with the therapeutic agent. Optimum results both economically and functionally, can often be obtained by filling only a portion of the hollow-core fibers, or partially filling some or all of the hollow-core fibers.

Combining the therapeutic agent with a hydrophobic material such as a solidifying agent; wax, solid ester, solid fatty alcohol or acid, hydrogenated vegetable oil, solid triglycerides, natural soft solid materials (i.e., cocoa butter), solid alkyl silicones, and the like, allows gradual diffusion of the active ingredient. In one embodiment, the solidifying agent can be solid at room temperature but can soften at body temperature to increase the release rate of the active ingredient once the product has been in contact with the body for a period of time. The active agent or other contents may be released from the hollows within the fibers by diffusion, vaporization, capillary action, liquid flow, liquid leakage, or other means. Where the active agent or other contents found in the hollow are in solid form, then the solid drugs may leave the hollow fiber by re-dissolving when in contact with new liquids and moving out by diffusion, capillary action, or a liquid flow.

Active ingredients can also be combined with an active deployment means that physically moves the active ingredient after being triggered by wetting or an increase in temperature. For example, the active deployment means can comprise generation of foam or bubbles in an effervescent effect that can move the active ingredient from within the absorbent article toward the body of the user, triggered by contact with an aqueous fluid, for example. A swellable material placed with the active ingredient in a pouch with a liquid-pervious inelastic wall can swell upon wetting and force expulsion of the active ingredient from the pouch.

The therapeutic agent may be combined into a formulation that may contain other additives or carrier components as appropriate for the desired result so long as the additives or carrier components do not have a major detrimental effect on the activity of the therapeutic agent. Examples of such additives include additional conventional surfactants, ethoxylated hydrocarbons, or co-wetting aids such as low molecular weight alcohols. The formulation is desirably applied from high solids, advantageously 80% or less solvent or water, so as to minimize drying and its attendant costs and deleterious effects. The treating formulation including a therapeutic agent may be applied in varying amounts depending on the desired results and application. Those skilled in the art can readily select the actual amount based on the teaching of this application. For example, a catamenial tampon designed to be inserted into a body cavity and subsequently in intimate contact with the vaginal epithelium may require substantially less therapeutic agent than an absorbent article worn exterior to the body due to the absence of first pass liver metabolism as previously discussed.

A formulation including a therapeutic agent may additionally employ one or more conventional pharmaceutically-acceptable and compatible carrier materials useful for the desired application. The carrier can be capable of co-dissolving or suspending the materials used in the formulation including a therapeutic agent. Carrier materials suitable for use in the instant formulation include those well-known for use in the pharmaceutical, cosmetic, and medical arts as a basis for ointments, lotions, creams, salves, aerosols, suppositories, gels and desirably are generally recognized as safe.

In yet another aspect of the present invention, the hollow-core fibers can be used to fabricate wound dressings and hemostat devices. There are several properties that wound dressing materials ideally should posses, such as the ability to remove excess exudate from the wound, protect the wound from mechanical injury, and reduce the risk of infection. Advantageously, the hollow-core fibers may be impregnated with such agents as alginates which can stimulate the clotting cascade for bleeding wounds, silver salts, antiseptics, and analgesics. The dressings can be used to treat a variety of wound types, such as infected wounds, incisions, punctures and burns. The advantage of using the hollow-core fibers of the present invention for such wound dressings is that the dressings are comfortable, flexible and absorbent and advantageously reduce the risk of infection absent the application or inclusion of specific antibiotics into the fiber core with respect to Staphylococcus aureus and Escherichia coli.

For convenience in explanation, embodiments are shown and described in this specification with fibers with one axial hollow. However, a fiber with multiple parallel axial hollows within each length of fiber can equally as well embody this invention. For example, fibers are currently made and can be used in the present invention that have one, four or seven hollows, respectively, running for the length of the fiber.

Although uses and commercial applications of the hollow-core fibers of the present invention have been shown and described as catamenial devices and bandages, other possible uses include diapers, incontinence articles, training pants, bed pads, sweat absorbing pads, skin cleansing pads, hard surface cleansing pads, shoe pads, helmet liners, absorbent devices for surgical and dental purposes (such as plugs for extracted teeth and saliva absorbents), air and water filtration devices, and industrial spill and leak absorbents.

Having described the invention in detail, those skilled in the art will appreciate that modifications may be made to the various aspects of the invention without departing from the scope and spirit of the invention disclosed and described herein. It is, therefore, not intended that the scope of the invention be limited to the specific embodiments illustrated and described but rather it is intended that the scope of the present invention be determined by the appended claims and their equivalents. Moreover, all patents, patent applications, publications, and literature references presented herein are incorporated by reference in their entirety for any disclosure pertinent to the practice of this invention.

I claim:

1. A hollow-core fiber comprising a phthalyl substituted cellulose ester polymer having a degree of substitution of phthalyl moieties of greater than about 0.13.

2. The hollow-core fiber of claim 1 wherein said degree of substitution of phthalyl moieties is from about 0.13 to about 1.3.

3. The hollow-core fiber of claim 1 wherein said degree of substitution of phthalyl moieties is from about 0.26 to about 1.3.

4. The hollow-core fiber of claim 1 wherein said degree of substitution of phthalyl moieties is from about 0.52 to about 1.04.

5. The hollow-core fiber of claim 1 wherein said degree of substitution of phthalyl moieties is from about 0.78 to about 1.04.

6. The hollow-core fiber of any one of claims 1 to 5 wherein the balance of said substituted acyl moieties is an ester having from two to twenty-four carbon atoms.

7. The hollow-core fiber of claim 6 wherein the balance of said substituted acyl moieties is one of more of acetate, butyrate, isobutyrate, or propionate.

8. A hollow-core fiber comprising a polymer blend comprising:
a. greater than about 75 mole % of a first polymer comprising a phthalyl substituted cellulose ester polymer having a degree of substitution of phthalyl moieties of greater than about 0.13; and
b. a second polymer or copolymer compatible with the first polymer, wherein the weight % of the first and second polymer equals 100.

9. The hollow-core fiber of claim 8 wherein said blend comprises greater than about 80 weight % of the first polymer and wherein the first polymer has a DS of phthalyl of from about 0.13 to about 1.3 and the balance of substituted acyl moieties being an ester having from two to twenty-four carbon atoms.

10. The hollow-core fiber of claim 9 wherein said blend comprises greater than about 85 weight % of the first polymer, and wherein the first polymer has a DS of from about 0.26 to about 1.3 and the balance of substituted acyl moieties are selected from acetate, butyrate, propionate, and mixtures thereof.

11. The hollow-core fiber of claim 9 wherein said blend comprises greater than about 95 weight % of the first polymer, and wherein the first polymer has a DS of from about 0.78 to about 1.04.

12. The hollow-core fiber of claim 1 or 8 wherein said fiber has an inner opening diameter of from about 5 to about 85 percent of the total diameter of hollow-core fiber.

13. The hollow-core fiber of claim 12 wherein said inner opening diameter is from about 25 to about 75 percent of the total diameter of hollow-core fiber.

14. The hollow-core fiber of claim 12 wherein said inner opening diameter is from about 50 to about 75 percent of the total diameter of hollow-core fiber.

15. The hollow-core fiber of claim 1 or 8 wherein said fiber has an anti-microbial effect.

16. The hollow-core fiber of claim 15 wherein said fiber is effective in reducing Staphylococcus aureus and Escherichia coli bacteria counts over a predetermined time period.

17. The hollow-core fiber of claim 15 wherein said fiber is effective in reducing Staphylococcus aureus bacteria CFU by more than 50% over a 24 hour period.

18. The hollow-core fiber of claim 1 wherein said fiber is effective in reducing Staphylococcus aureus bacteria CFU by more than 90% over a 24 hour period.

19. The hollow-core fiber of claim 1 or 8 further comprising an auxiliary compound within the open area of said fiber.

20. The hollow-core fiber of claim 19 wherein said auxiliary compound is one or more of absorbents, odor controlling or absorbing materials, fragrances and perfumes, medicaments, or therapeutic agents.

21. The hollow-core fiber of claim 20 wherein said absorbent is a superabsorbent; said fragrances and perfumes are one or more of volatile hydrophilic materials, volatile hydrophobic materials, or low odor detection threshold perfume materials; said medicaments and therapeutic agents are one or more of histidines, anti-inflammatory drugs, calcium or potassium channel blockers, antimicrobials, anti-viral agents, antifungal agents, anti-metabolites, steroids, anesthetics, hormones or hormone inhibitors, pH control agents, vitamins, moisturizers, or pro-biotic agents.

22. An absorbent article comprising a fluid transfer member and a fluid storage member, wherein said fluid transfer member comprises from about 5 to 100 weight % of at least one hollow-core fiber comprising:
a. a phthalyl substituted cellulose ester polymer having a degree of substitution of phthalyl moieties of greater than about 0.13; or
b. a polymer blend comprising:
i. greater than about 75 mole % of a first polymer comprising a phthalyl substituted cellulose ester polymer having a degree of substitution of phthalyl moieties of greater than about 0.13; and
ii. a second polymer or copolymer compatible with the first polymer, wherein the weight % of the first and second polymer equals 100.

23. The absorbent article of claim 22 wherein the phthalyl substituted cellulose ester polymer has a degree of substitution of from about 0.13 to about 1.3, and a balance of the substituted acyl moieties is an ester having from two to twenty-four carbon atoms.

24. The absorbent article of claim 23 wherein the phthalyl substituted cellulose ester polymer has a degree of substitution of from about 0.26 to about 1.3, and the balance of the substituted acyl moieties is one or more of acetate, butyrate, or propionate.

25. The absorbent article of claim 23 wherein the phthalyl substituted cellulose ester polymer has a degree of substitution of from about 0.52 to about 1.04.

26. The absorbent article of claim 23 wherein the phthalyl substituted cellulose ester polymer has a degree of substitution of from about 0.78 to about 1.04.

27. The absorbent article of claim 23 wherein the fluid transfer member comprises from about 50 to 100 weight % of the hollow-core fiber.

28. The absorbent article of claim 23 wherein the fluid transfer member comprises from about 95 to 100 weight % of the hollow-core fiber.

29. The absorbent article of claim 23 wherein the hollow-core fiber further comprises a secondary compound within a portion of the fiber which is one or more of absorbents, odor controlling or absorbing materials, fragrances and perfumes, medicaments, or therapeutic agents.

30. The absorbent article of claim 23 wherein said absorbent article comprises catamenial devices, bandages, diapers, incontinence articles, training pants, bed pads, sweat absorbing pads, skin cleansing pads, hard surface cleansing pads, shoe pads, helmet liners, absorbent devices for surgical and dental purposes, air and water filtration devices, or industrial spill and leak absorbents.
